(19) United States
(12) Patent Application Publication
(10) Pub. No.: US 2019/0059737 A1
(43) Pub. Date: Feb. 28, 2019

(54) IMAGING METHOD USING FLUOROQUINOLONE ANTIBIOTICS AND IMAGING DEVICE FOR THE SAME

(71) Applicant: POSTECH ACADEMY-INDUSTRY FOUNDATION, Pohang-si (KR)

(72) Inventors: Seunghun LEE, Daegu (KR); Ki Hean Kim, Pohang-si (KR); Jun Ho Lee, Seoul (KR); Bumju Kim, Pohang-si (KR); Byung Ho Oh, Seoul (KR); Myoung Joon Kim, Seoul (KR); Kyo Han Anh, Pohang-si (KR); Hoon Cheol Jang, Seoul (KR); Won Hyuk Jang, Seongnam-si (KR)

(21) Appl. No.: 15/893,877

(22) Filed: Feb. 12, 2018

(30) Foreign Application Priority Data
Aug. 31, 2017 (KR) 10-2017-0110974

(51) Int. Cl. A61B 5/00; A61B 1/00; A61B 1/06; A61B 1/313; A61B 3/10; A61B 5/08; A61B 5/20; A61K 49/00

(52) U.S. Cl. CPC A61B 5/0071; A61K 49/0052; A61B 1/00186; A61B 1/0623; A61B 1/3132; A61B 3/10; A61B 5/0064; A61B 5/0088; A61B 5/08; A61B 5/4233; A61B 5/4238; A61B 5/4244; A61B 5/425; A61B 5/4255; A61B 5/20; A61B 1/00172

(57) ABSTRACT

Disclosed are an imaging method using fluoroquinolone antibiotics and an imaging device for the same, in which biological tissue is stained with Moxifloxacin as one of fluoroquinolone antibiotics, and the stained biological tissue is subjected to fluorescent image-capture through single-photon excitation with either near-ultraviolet or visible wavelength light instead of either a middle-ultraviolet light source or a femtosecond near-infrared laser device, thereby obtaining morphological information of cells in the biological tissue at a high speed without damage. To this end, an imaging method of using fluoroquinolone antibiotics includes: staining cells of the biological tissue with fluoroquinolone antibiotics; illuminating the excitation light from a light source to the biological tissue stained with the fluoroquinolone antibiotics; and capturing an image of the biological tissue through the fluoroquinolone antibiotics based fluorescence caused by the excitation light illuminated to the biological tissue, wherein the excitation light from the light source includes either near-violet or short visible wavelength light for single photon excitation of the fluoroquinolone antibiotics.

### CROSS-REFERENCE TO RELATED APPLICATION

This application claims priority from Korean Patent Application No. 10-2017-0110974, filed on Aug. 31, 2017 in the Korean Intellectual Property Office, the disclosure of which is incorporated herein by reference.

### ACKNOWLEDGEMENTS

This research was supported by the projects as below.

[Project number] 2016928790

[Ministry] Ministry of Science and ICT

[Management agency] National Research Foundation of Korea

[Program name] Scientific Research Center/Engineering Research Center Foster Project

[Project name] Research Center for Advanced Robotic Surgery based on Deep Tissue Imaging and Haptic Feedback Technology

[Contribution ratio] 30%

[Supervision institution] POSTECH Research and Business Development Foundation

[Period] Sep. 1, 2016-Aug. 31, 2017

[Project number] 2017044964

[Ministry] Ministry of Science and ICT

[Management Agency] National Research Foundation of Korea

[Program name] Brain Research Program

[Project name] Development of High-speed 3D Fluorescence Microscope Systems for Comprehensive Molecular Imaging of Optical Cleared Mouse Brains

[Contribution ratio] 30% [Supervision institution] POSTECH Research and Business Development Foundation

[Period] Jun. 1, 2017-Feb. 28, 2018

[Project number] POSTECH internal project

[Project name] Development of Moxifloxacin Based Three-photon Microscopy for Clinical Research

[Contribution ratio] 40%

[Supervision institution] POSTECH Research and Business Development Foundation

[Period] Mar. 1, 2017-Feb. 28, 2018

### BACKGROUND OF THE INVENTION

### (a) Field of the Invention

The present disclosure relates to an imaging method of cells in the biological tissue by using fluoroquinolone antibiotics and an imaging device for the same, and more particularly to an imaging method using fluoroquinolone antibiotics and an imaging device for the same, in which cells in the biological tissue is stained with Moxifloxacin as one of fluoroquinolone antibiotics, and the stained biological tissue is subjected to fluorescent image-capture through single-photon excitation by using either a near-ultraviolet or short visible wavelength light source instead of either a middle-ultraviolet light source or a near-infrared femtosecond laser device, thereby obtaining cellular information of the biological tissue at a high speed without damage.

### (b) Description of the Related Art

Optical microscopy capable of capturing images of cells in the biological tissue at a high resolution has been used in study of biology, and utilized in a skin diagnosis in a clinic.

Optical fluorescence microscopy employs autofluorescence of the biological tissue in case of not using exogenous fluorescent probes and thus label-free optical fluorescence microscopy has problems of low contrasts and low imaging speeds due to weak autofluorescence. Therefore, the biological tissue is generally stained with fluorescent probes, and then the optical fluorescence microscopy can capture high contrast images of cells in the biological tissue at high speeds.

In terms of utilizing the fluorescent probe, various kinds of fluorescent probe are used in animal model based studies, but only a few fluorescent probes such as indocyanine green and fluorescein are used for the human as fluorescent probes to stain the blood vessel.

Further, only the stained blood vessel is not enough to diagnose lesions or cancers or to obtain morphological information of cells, and therefore cell staining in the human body is needed for making an accurate diagnosis.

To this end, fluorescent-staining medical substances have been studied for the cell staining aimed at the human body, but there are no fluorescent probes suitable for the human body at this point in time because of toxicity or the like problem.

Among the medical substances for the cell staining, Moxifloxacin, which is an antibiotic used to either treat or prevent bacterial infections in a current clinic, has been demonstrated as a cell labeling agent in the biological tissue for two-photon microscopy which is a fluorescent imaging method using a femtosecond light source. Moxifloxacin is known to have an intrinsic fluorescence property and good tissue penetration properties. Through optical microscopic imaging studies, moxifloxacin was found to be used as a cell labeling agent owing to its high distribution inside the cells rather than outside the cells in the tissue.

Moxifloxacin has its maximum absorption spectrum at a middle-ultraviolet region of 280 nm, and ultraviolet rays are harmful to the human body. To solve this problem, fluorescent imaging of the biological tissue based on two-photon excitation of Moxifloxacin using near infrared excitation light has recently been demonstrated, and it is ascertained that the forms of the in-vivo tissue and cells stained with Moxifloxacin can be imaged at a high resolution and a high speed.

However, since the two-photon excitation is a nonlinear process and high excitation light density is needed to generate the nonlinear two-photon excitation, the two-photon excitation requires an expensive femtosecond laser device.

Such a femtosecond laser device typically costs approximately 50 to 150 million Korean won, which is very expensive as compared with a general continuous wave (CW) laser device that costs approximately 10 million Korean won. In case of using the femtosecond laser device, commercialization is difficult since costs of equipment for taking a imaging are high.

Further, the two-photon excitation usually has a relatively low excitation-efficiency compared to typical single photon excitation, and thus causes a problem that the imaging speed of Moxifloxacin-based two-photon microscopy is not high enough.

To solve this problem, higher excitation light power may be used. However, this may cause problems that there is a limit to the excitation light power of the laser device and the higher excitation light power may damage the biological tissue.

### SUMMARY OF THE INVENTION

Accordingly, the present disclosure is conceived to solve the conventional problems, and an aspect of the present disclosure is to provide an imaging method using fluoroquinolone antibiotics and an imaging device for the same, in which biological tissue is stained with Moxifloxacin as one of fluoroquinolone antibiotics, and the stained biological tissue is subjected to fluorescence imaging through single-photon excitation at either a near-ultraviolet or a visible excitation wavelength without using a femtosecond laser device, thereby obtaining cellular information of the biological tissue at a high speed and a low equipment cost.

Moxifloxacin is known to have its maximum absorption at middle-ultraviolet wavelength. Our further measurement showed that the excitation maximum of Moxifloxacin was at near-ultraviolet wavelength and Moxifloxacin could be excited even by using visible excitation wavelength as well. This indicates that Moxifloxacin based fluorescent imaging of cells in the biological tissue can be possible by using either near-ultraviolet or visible wavelength without damage instead of middle-ultraviolet.

According to an aspect of the present disclosure, there is provided an imaging method of using fluoroquinolone antibiotics, the method including: staining cells in the biological tissue with fluoroquinolone antibiotics; illuminating light from a light source to the biological tissue stained with the fluoroquinolone antibiotics; and capturing an image of the biological tissue through the fluorescent light of fluoroquinolone antibiotics caused by the light illuminated to the biological tissue, wherein the light from the light source is either near-ultraviolet or short visible wavelength for single photon excitation of the fluoroquinolone antibiotics.

The fluoroquinolone antibiotics may include Moxifloxacin.

The light from the light source may have a continuous wave wavelength range including a near-ultraviolet region and a visible region.

The near-ultraviolet wavelength and the short visible wavelength of the light emitted from the light source may range from 300 nm to 476 nm.

The biological tissue may include at least one of external organs, and internal organs, which can be subjected to endoscopy and laparoscopy, of a human body.

The external organs may include at least one among the cornea, skin and tongue, and the internal organs may include at least one among the small intestine, large intestine, stomach, bladder, brain, lung, esophagus, liver, and pancreas.

The image capturing of the biological tissue may include: a photon moving operation in which fluorescent light of the fluoroquinolone antibiotics generated by the light illuminated to the biological tissue is moved to a light detector; a photon collecting operation in which the fluorescent light moved to the light detector is collected at the light detector; a photo signal processing operation in which the fluorescence collected at the light detector is subjected to a signal process in a data driving/obtaining board so as to be output through an output section; and a photon outputting operation in which a fluorescent signal processed in the photon signal processing operation is output through the output section.

According to an aspect of the present disclosure, there is provided an imaging device including: a light source configured to illuminate light to biological tissue stained with fluoroquinolone antibiotics; and a variable neutral density (ND) filter configured to control the amount of light from the light source; a scanner configured to adjust an angle at which light from the light source or fluorescence of the fluoroquinolone antibiotics excited by the light is reflected; a dichroic mirror configured to transmit or reflect light in accordance with wavelengths of the light; a lens configured to control a path via which the light from the light source or the fluorescent light of the fluoroquinolone antibiotics excited by the light travels; a light detector configured to collect the fluorescent light of fluoroquinolone antibiotics; a data driving/obtaining board configured to perform a signal process to output the fluorescence collected at the light detector; and an output section configured to output the fluorescence processed in the data driving/obtaining board, wherein the light from the light source includes light for single photon excitation of the fluoroquinolone antibiotics.

The fluoroquinolone antibiotics may include Moxifloxacin.

The light from the light source may have a continuous wave wavelength range including the near-ultraviolet region and short visible region.

The near-ultraviolet region and the short visible region of the light from the light source may range from 300 nm to 476 nm.

### BRIEF DESCRIPTION OF THE DRAWINGS

The above and/or other aspects of the present disclosure will become apparent and more readily appreciated from the following description of the exemplary embodiments, taken in conjunction with the accompanying drawings, in which:

FIGS. 1(a) and 1(b) illustrate a mechanism of single photon excitation and two-photon excitation, in an imaging method using fluoroquinolone antibiotics according to the present disclosure and an imaging device for the same;

FIGS. 2(a) and 2(b) illustrate a single photon excitation spectrum and a fluorescence emission spectrum in near-ultraviolet and short visible regions of Moxifloxacin used in an imaging method using fluoroquinolone antibiotics according to the present disclosure and an imaging device for the same;

FIG. 3 illustrates a 405 nm laser-based imaging device for capturing an image of biological tissue, in an imaging method using fluoroquinolone antibiotics according to the present disclosure and an imaging device for the same;

FIGS. 4(a) and 4(b) are photographs of the mouse's small intestine tissue not stained with Moxifloxacin, obtained by the fluorescent image-capture device based on the single photon excitation with a 405 nm continuous wave laser. FIGS. 4(c) and 4(d) are photographs of the mouse's small intestine tissue stained with Moxifloxacin, obtained by the fluorescent image-capture based on single photon excitation with a 405 nm continuous wave laser. FIG. 4(e) is a graph of showing results from quantitatively analyzing fluorescence intensity of the mouse's small intestine tissue obtained by the single-photon excitation-based fluorescent image-capture using the continuous wave laser light of 405 nm without and with Moxifloxacin staining, in an imaging method using fluoroquinolone antibiotics according to the present disclosure and an imaging device for the same;

FIGS. 5(a) and 5(b) are photographs of the mouse large intestine tissue stained with Moxifloxacin, obtained by the fluorescent image-capture based on the single photon excitation with a 405 nm continuous wave laser, in an imaging method using fluoroquinolone antibiotics according to the present disclosure and an imaging device for the same;

FIGS. 6(a) and 6(b) are photographs of the mouse stomach tissue stained with Moxifloxacin, obtained by the fluorescent image-capture based on the single photon excitation with a 405 nm continuous wave laser in an imaging method using fluoroquinolone antibiotics according to the present disclosure and an imaging device for the same;

FIGS. 7(a), 7(b) and 7(c) are photographs of the mouse bladder tissue stained with Moxifloxacin, obtained by the fluorescent image-capture based on the single photon excitation with a 405 nm continuous wave laser in an imaging method using fluoroquinolone antibiotics according to the present disclosure and an imaging device for the same;

FIGS. 8(a), 8(b), 8(c) and 8(d) are photographs of the mouse cornea tissue stained with Moxifloxacin, obtained by confocal reflectance microscopy. FIG. 8(e)˜(h) are photographs of the same mouse corneas as that of FIG. 8(a)˜(d), obtained by the fluorescent image-capture based on the single photon excitation with a 405 nm continuous wave laser in an imaging method using fluoroquinolone antibiotics according to the present disclosure and an imaging device for the same; and

FIG. 9 is a flowchart of an imaging method using fluoroquinolone antibiotics according to the present disclosure.

### DETAILED DESCRIPTION OF THE EMBODIMENTS

Hereinafter, embodiments for materializing the foregoing aspect of the present disclosure will be described with reference to the accompanying drawings. In describing the embodiments, like numerals refer to like elements, and repetitive descriptions thereof will be avoided as necessary.

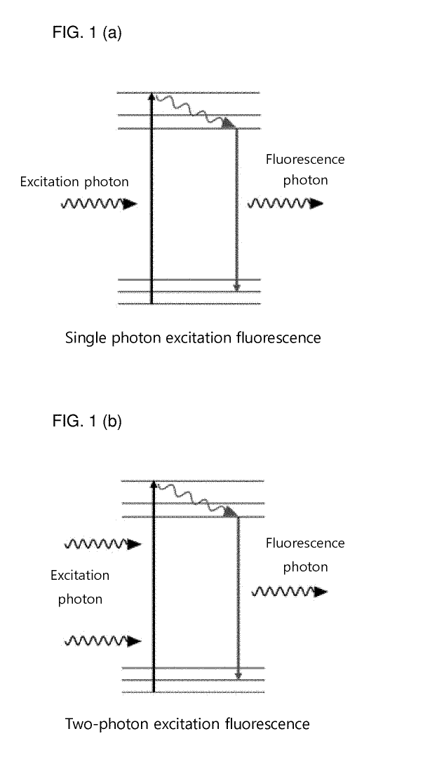

Referring to FIG. 1 to FIG. 9, an imaging method using fluoroquinolone antibiotics according to the present disclosure and an imaging device for the same will be described below.

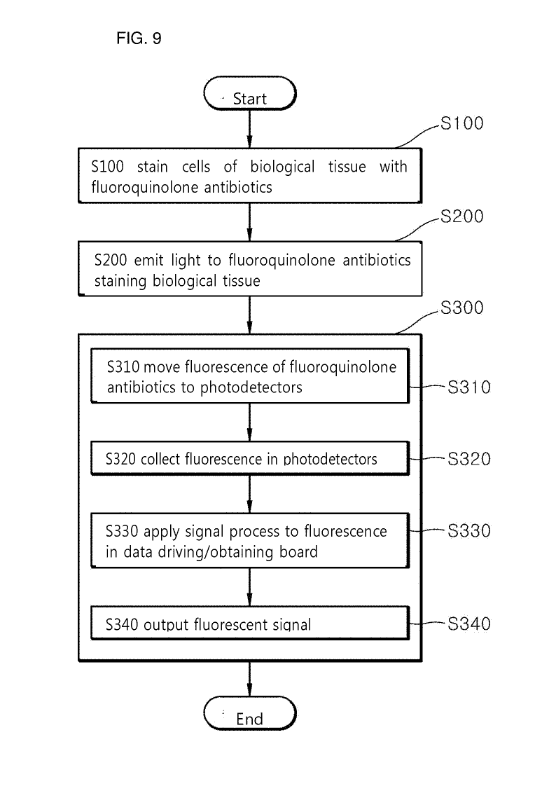

As shown in FIG. 9, an imaging method using fluoroquinolone antibiotics according to the present disclosure includes operations of staining biological tissue (S100), emitting light to the biological tissue (S200), and capturing an image of the biological tissue (S300), in which an imaging device is used as a confocal fluorescence microscopy assembly for capturing the image of the biological tissue.

Before describing the imaging method using fluoroquinolone antibiotics according to the present disclosure, a mechanism of single photon excitation and two-photon excitation will be first described with reference to FIG. 1.

As shown in FIG. 1, an excitation photon makes an energy level transition of an electron in fluorescent molecules from a ground state to an excited state. Then, a fluorescence photon is emitted when the energy level of the electron goes back down from the excited state to the ground state.

A phenomenon of absorbing one excitation photon and absorbing one fluorescence photon as shown in FIG. 1(a) is called single photon excitation, and a phenomenon of absorbing two excitation photons and emitting one fluorescence photon as shown in FIG. 1(b) is called two-photon excitation.

With this, when molecules, cells and tissue of an organism are treated with a fluorescent probe, the activities of the molecules, cells and tissue of the organism are observable at high resolution through optical fluorescence microscopy. This is possible because a fluorescence photon of a certain color is emitted while an electron in the fluorescent probe, jumped up to the excited state by the excitation photon, goes back to the ground state.

When the fluorescent probe is introduced into biological tissues, absorbed in cells of the biological tissue, and maintained at high concentration, the biological tissue can be imaged with high contrast using the fluorescent probe.

In this case, the fluorescent probe is usable for staining the biological tissue to obtain morphological information of the biological tissue under conditions that the fluorescent probe of staining the biological tissue is not harmful to a human body and fluorescence excitation is possible in a visible region where there are no negative effects on a human body.

According to the present disclosure, the fluoroquinolone antibiotics used for staining the biological tissue include Moxifloxacin, Gatifloxacin, Pefloxacin, Difloxacin, Norfloxacin, Ciprofloxacin, Ofloxacin, Enrofloxacin, etc. In this specification, Moxifloxacin, which can generate strong fluorescence in the visible region, is used to stain the biological tissue.

According to the present disclosure, it is ascertained that the imaging device employing a confocal fluorescence microscopy assembly can capture images of cells in the biological tissue through single photon excitation fluorescence of Moxifloxacin, and it is ascertained that an inexpensive light source is also usable to capture images of cells in the biological tissue through Moxifloxacin since the imaging device originally employs a continuous wave light source worth about 10 million won to perform fluorescent image-capture through the single photon excitation of Moxifloxacin.

Of course, the imaging method using fluoroquinolone antibiotics according to the present disclosure is performed by the imaging device of the optical microscopy assembly that uses the continuous wave light source to generate the single photon excitation fluorescence of Moxifloxacin and captures an image. Alternatively, other kinds of optical microscopy may be also available aside from the imaging device.

Therefore, it is possible to provide a method of capturing images of cells in a human body through an inexpensive image-capturing system based on the single photon excitation fluorescence of Moxifloxacin since the single photon excitation efficiency is higher than two-photon excitation efficiency.

According to experimental examples of the present disclosure, Moxifloxacin was injected into small intestine cells, large intestine cells, stomach cells, bladder cells, and cornea cells, and thus fluorescence generation in each cell and the morphological information of the cell were ascertained.

Further, the imaging method using fluoroquinolone antibiotics according to the present disclosure is applicable to capture images of small intestine, large intestine, stomach, bladder, lung, esophagus, liver, brain, pancreas, and the like internal organs, which can be subjected to endoscopy and laparoscopy, as well as cornea, skin, tongue, and the like external organs.

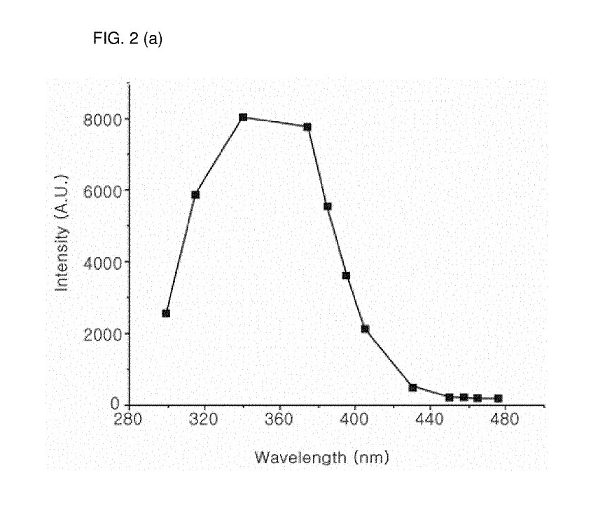

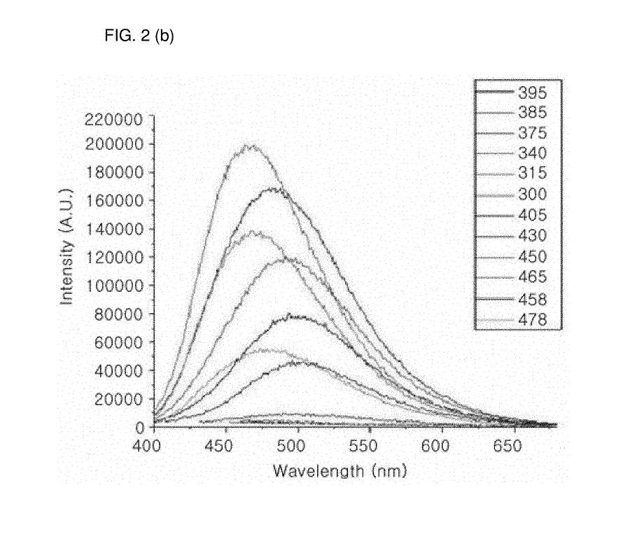

Further, a single photon excitation spectrum and a fluorescence emission spectrum of Moxifloxacin will be described below with reference to FIG. 2.

FIG. 2(a) and FIG. 2(b) respectively show the excitation spectrum and the fluorescence emission spectrum of Moxifloxacin in the near-ultraviolet region and the visible region.

For the imaging method using fluoroquinolone antibiotics according to the present disclosure, Vigamox ophthalmic solution of 0.5% (Alcon, the U.S.) on the market was used as Moxifloxacin

As shown in FIG. 2, Moxifloxacin had the highest excitation efficiency at a wavelength of about 340 nm in the near-ultraviolet region, and was then gradually decreased in excitation efficiency as the wavelength increases.

However, Moxifloxacin was also excited at wavelengths of 405 nm to 478 nm in the visible region outside the near-ultraviolet range, and had fluorescence intensity of about 27% at a wavelength of 405 nm as compared with that at a wavelength of 340 nm. This fluorescence intensity is much higher than the fluorescence intensity of two-photon excitation at a wavelength of 700 nm.

Therefore, the continuous wave light source used in the imaging method using fluoroquinolone antibiotics according to the present disclosure can use light of wavelengths of 300 nm to 476 nm, i.e. near-ultraviolet to middle-ultraviolet wavelengths to thereby increase the fluorescent signals and capture images at higher speed. Further, wavelengths corresponding to short visible light are also applicable to the biological tissue.

Among the single photon wavelengths, the near-ultraviolet wavelengths may cause cell damage in the in-vivo tissue, and thus be used in capturing an image of biological tissue excised during an operation or the like.

Further, among the single photon wavelengths, the wavelengths corresponding to the visible light have a sufficient excitation efficiency without causing any cell damage in the in-vivo tissue, and are thus usable in capturing images of all kinds of biological tissue.

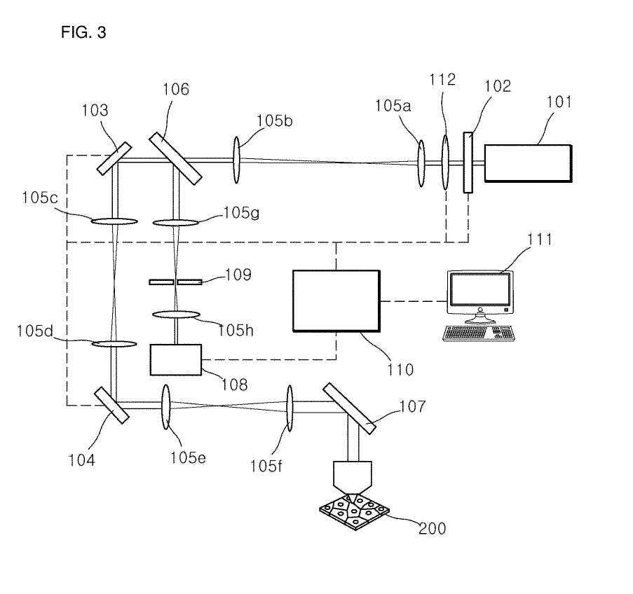

An imaging device for capturing an image of biological tissue by the imaging method using fluoroquinolone antibiotics according to the present disclosure will be described below with reference to FIG. 3.

As shown in FIG. 3, the imaging device according to the present disclosure includes a light source 101, a shutter 102, an X-axis scanner 103, a Y-axis scanner 104, a lens 105, a dichroic mirror 106, a reflection mirror 107, a light detector 108, a pin hole 109, a data driving/obtaining board 110, and a computer 111. The lens 105 includes a first lens 105a, a second lens 105b, a third lens 105c, a fourth lens 105d, a fifth lens 105e, a sixth lens 105f, a seventh lens 105g and an eighth lens 105h in accordance with positions.

Using the imaging device, the imaging method using fluoroquinolone antibiotics according to the present disclosure will be described below.

In the operation S100 of staining the biological tissue, cells of the biological tissue to be subjected to an experiment are stained with fluoroquinolone antibiotics, in which Moxifloxacin is used as the fluoroquinolone antibiotics according to the present disclosure.

In the operation S200 of emitting light to the biological tissue, the light source 101 emits light to Moxifloxacin of staining the biological tissue. Here, the light source 101 is capable of emitting continuous wave light of the near-ultraviolet and visible ranges. In the following experimental examples, the light source 101 emits continuous wave laser light having a wavelength of 405 nm for the experiment.

As shown in FIG. 3, in the operation S200 of emitting light to the biological tissue, a single photon sequentially passes through the light source 101, the shutter 102, a variable neutral density (ND) filter 112, the first lens 105a, the second lens 105b, the dichroic mirror 106, the X-axis scanner 103, the third lens 105c, the fourth lens 105d, the Y-axis scanner 104, the fifth lens 105e, the sixth lens 105f, the reflection mirror 107, an object lens 105, and biological tissue 200, so that the single photon output from the light source 101 can be emitted to the biological tissue 200.

Here, the variable ND filter 112 is a light blocking filter having a neutral characteristic with regard to colors, in which a penetration amount of light having a certain wavelength within a specific wavelength range varies, thereby controlling the penetration amount.

Further, in the operation S200 of emitting light to the biological tissue, the single photon passes through the dichroic mirror 106 and moves to the X-axis scanner 103. Here, the dichroic mirror 106 passes or reflects light in accordance with the wavelength of the light.

In the operation S300 of capturing an image of the biological tissue, the image of the biological tissue is captured through the fluorescence excitation of Moxifloxacin, caused by the light emitted to the biological tissue in the operation S200. The operation S300 of capturing an image of the biological tissue includes a photon moving operation S310, a photon collimating operation S320, a photon signal processing operation S330, and a photon outputting operation S340.

In the photon moving operation S310, the fluorescence of Moxifloxacin caused by the light emitted to the biological tissue in the operation S200 moves to the light detector 108. As shown in FIG. 3, the photon in the photon moving operation S310 sequentially moves via the biological tissue 200, the reflection mirror 107, the sixth lens 105f, the fifth lens 105e, Y-axis scanner 104, the fourth lens 105d, the third lens 105c, X-axis scanner 103, the dichroic mirror 106, the seventh lens 105g, a pin hole 109, the eighth lens 105h and the light detector 108.

On the contrary to the light emitted to the biological tissue in the operation S200, the photon in the photon moving operation S310 is reflected from the dichroic mirror 106 and moves to the seventh lens 105g.

In the photon collimating operation S320, the photon moved in the photon moving operation S310 is collected at the light detector 108, thereby maximizing an output fluorescent signal.

In the photon signal processing operation S330, the fluorescence collected at the light detector 108 is subjected to a signal process in the data driving/obtaining board 110 and then output through an output section.

In the photon outputting operation S340, a fluorescent signal processed in the photon signal processing operation S330 is output from the output section. This means that the morphological information of the biological tissue is output. The output section refers to the computer 111. Through a Lab-view program coded in the computer 111, the control and output of the data driving/obtaining board 110 may be performed.

Further, the data driving/obtaining board 110 may control and drive the X-axis scanner 103, the Y-axis scanner 104, the shutter 102, and the variable ND filter 112.

Further, in the imaging method using fluoroquinolone antibiotics according to the present disclosure, the light source 101 may use not only the continuous wave laser device but also a light emitting diode or a discharge lamp to emit light, thereby capturing the image of the biological tissue.

The foregoing imaging method using fluoroquinolone antibiotics according to the present disclosure will be described below with reference to experimental examples.

### EXPERIMENTAL EXAMPLE 1

### Image Capture of Cells in a Mouse's Small Intestine Tissue Based on Single Photon Fluorescence Excitation of Moxifloxacin

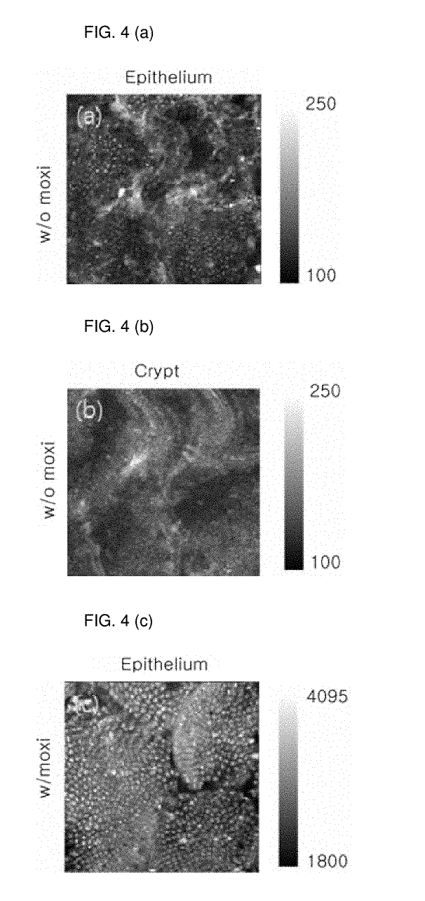

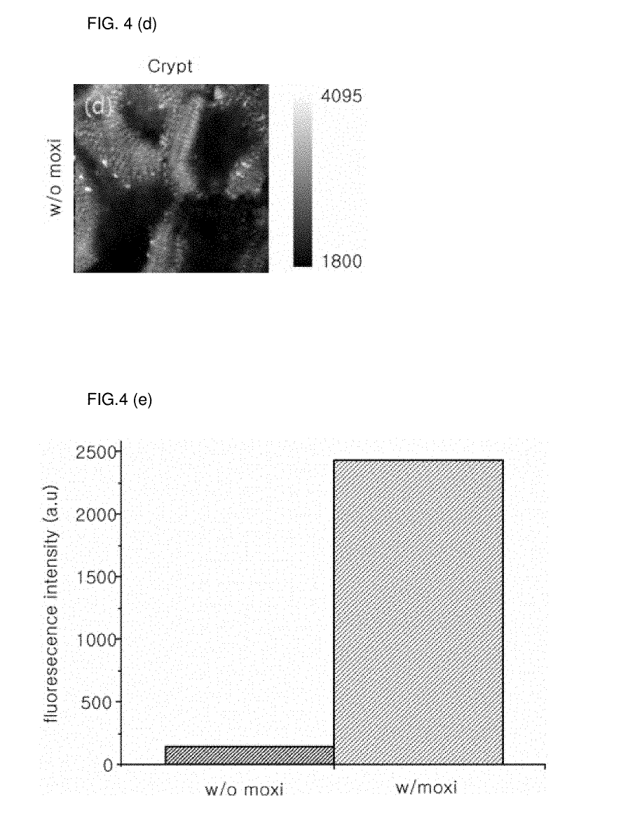

FIGS. 4(a) and (b) are photographs obtained from Moxifloxacin fluorescence image-capture using the single photon excitation caused by emitting the continuous wave laser light having a wavelength of 405 nm in the imaging device of FIG. 3 to a lumen of a mouse's small intestine before injecting Moxifloxacin, FIGS. 4(c) and (d) are photographs obtained from Moxifloxacin fluorescence image-capture using the single photon excitation caused by emitting the continuous wave laser light having a wavelength of 405 nm to a lumen of a mouse's small intestine after injecting Moxifloxacin, and FIG. 4(e) is is a graph of showing results from quantitatively analyzing fluorescence intensity of a mouse's small intestine tissue obtained by the single-photon excitation-based fluorescent image-capture using the continuous wave laser light of 405 nm before and after being stained with Moxifloxacin.

Here, FIGS. 4(a) and (c) show an epithelium of a mouse's small intestine, and FIGS. 4(c) and (d) show a villus of a mouse.

As shown in FIG. 4, autofluorescence of cells in a mouse's small intestine tissue was so weak that cell image capture was difficult with low contrast unless Moxifloxacin is injected. On the other hand, after injecting Moxifloxacin, the cells of the epithelium and villus were imaged with high contrast at high resolution due to the single photon excitation fluorescence of Moxifloxacin.

That is, it was ascertained that Moxifloxacin generated fluorescence stronger than the autofluorescence while being maintained at high concentration in the biological tissue cells of the small intestine. As shown in FIG. 4(e), it was ascertained that the single photon excitation fluorescence of Moxifloxacin is stronger eighteen times than the autofluorescence. Accordingly, it is possible to obtain the morphological information with high contrast.

### EXPERIMENTAL EXAMPLE 2

### Image Capture of Cells in a Mouse's Large Intestine Tissue Based on Single Photon Fluorescence Excitation of Moxifloxacin

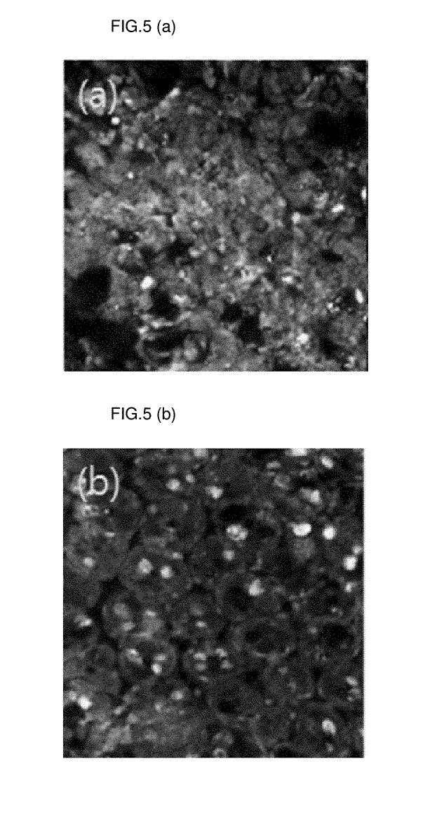

FIGS. 5(a) and (b) are photographs obtained from Moxifloxacin fluorescence caused by the single photon excitation when the continuous wave laser light of 405 nm is emitted from the imaging device of FIG. 3 to a lumen of a mouse's large intestine stained with Moxifloxacin.

Here, FIG. 5(a) shows epithelium of a mouse's small intestine, and FIG. 5(b) shows crypt of a mouse.

As shown in FIG. 5, the epithelial cells of the epithelium and the goblet cells of the intestinal crypt were imaged at high resolution due to the single photon excitation fluorescence of Moxifloxacin after injecting Moxifloxacin.

That is, it was ascertained that Moxifloxacin generated fluorescence stronger than the autofluorescence while being maintained at high concentration in the biological tissue cells of the large intestine. Accordingly, it is possible to obtain the morphological information of a mouse's large intestine with high contrast.

### EXPERIMENTAL EXAMPLE 3

### Image Capture of Cells in a Mouse's Stomach Tissue Based on Single Photon Fluorescence Excitation of Moxifloxacin

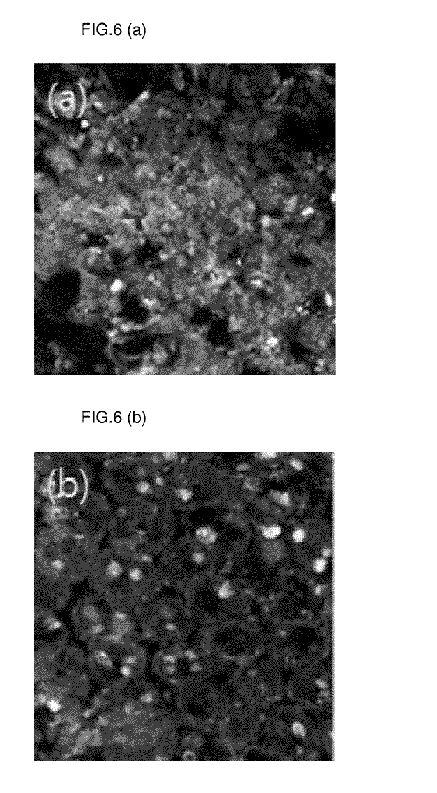

FIG. 6 is a photograph obtained from Moxifloxacin fluorescence caused by the single photon excitation when the continuous wave laser light of 405 nm is emitted from the imaging device of FIG. 3 to a lumen of a mouse's stomach stained with Moxifloxacin.

Here, FIG. 6(a) shows epithelium of a mouse's stomach, and FIG. 6(b) shows crypt of a mouse's stomach.

As shown in FIG. 6, the cells in the epithelium and crypt of a mouse's stomach were imaged at high resolution due to the single photon excitation fluorescence of Moxifloxacin, and it was thus ascertained that Moxifloxacin generated strong fluorescence while being maintained at high concentration in the biological tissue cells of the stomach.

### EXPERIMENTAL EXAMPLE 4

### Image Capture of Cells in a Mouse's Bladder Tissue Based on Single Photon Fluorescence Excitation of Moxifloxacin

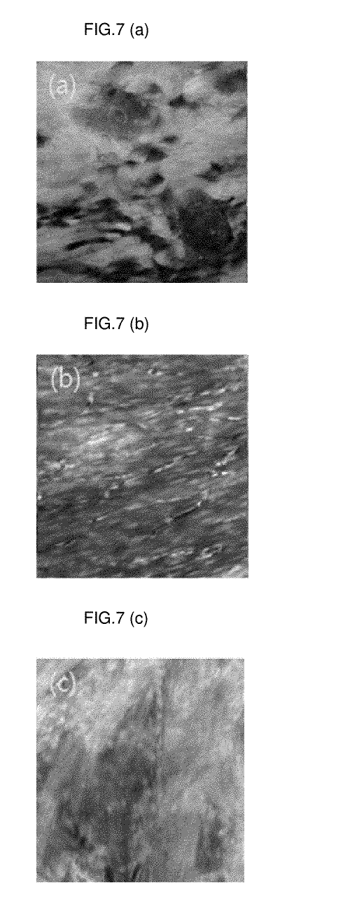

FIG. 7 is a photograph obtained from Moxifloxacin fluorescence caused by the single photon excitation when the continuous wave laser light having a wavelength of 405 nm is emitted from the imaging device of FIG. 3 to a lumen of a mouse's bladder stained with Moxifloxacin.

Here, FIG. 7(a) shows uroepithelium of a mouse's bladder epithelium, and FIG. 7(b) shows lamina propria of a mouse's bladder, FIG. 7(c) shows a muscle layer of a mouse's bladder.

As shown in FIG. 7, umbrella cells of the uroepithelium of a mouse's bladder, vascular endotheliocytes of the lamina propria, and muscle of the muscle layer were imaged at high resolution due to the single photon excitation fluorescence of Moxifloxacin.

That is, it was ascertained that Moxifloxacin generated strong fluorescence while being maintained at high concentration in the biological tissue cells of the bladder.

### EXPERIMENTAL EXAMPLE 5

### Comparison Between Image Capture of Cells in a Mouse's Cornea Tissue Based on Single Photon Fluorescence Excitation of Moxifloxacin using the Imaging Device and Image Capture Based on Confocal Reflectance Microscopy

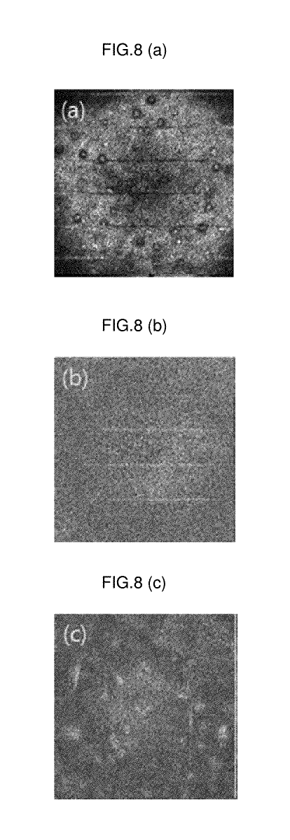

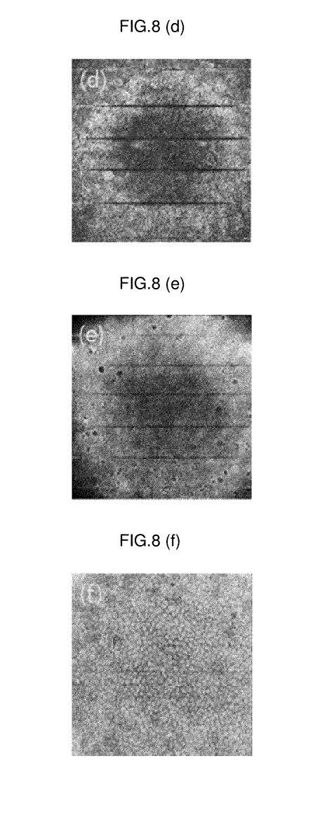

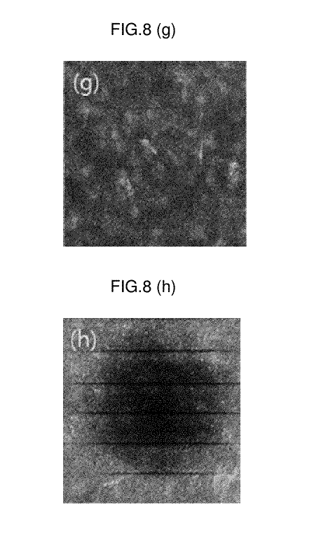

FIG. 8(a)˜(d) are photographs obtained applying confocal reflectance to a mouse's cornea stained with Moxifloxacin, and FIGS. 8(e)˜(h) are photographs obtained from Moxifloxacin fluorescence caused by the single photon excitation when the continuous wave laser light having a wavelength of 405 nm is emitted from the imaging device of FIG. 3 to the same cornea as that of FIG. 8(a)˜(d).

Here, FIGS. 8(a) and (e) show superficial epithelium of a mouse's cornea, FIGS. 8(b) and (f) show basal epithelium, FIGS. 9(c) and (g) show corneal stroma, and FIGS. 8(d) and (h) show corneal endothelium.

As shown in FIG. 8, corneal epithelial cells of the superficial epithelium were imaged at high resolution in FIGS. 8(a), (b), (e) and (f), keratocytes of the basal epithelium were imaged at high resolution in FIGS. 8(c) and (g), and corneal endotheliocytes of the endothelium were imaged at high resolution in FIGS. 8(d) and (h).

That is, it was ascertained that Moxifloxacin generated strong fluorescence while being maintained at high concentration in the biological tissue cells of the cornea.

The imaging method using fluoroquinolone antibiotics according to the present disclosure, and an imaging device for the same have effects as follows.

First, the biological tissue is stained with one of fluoroquinolone antibiotics, i.e. Moxifloxacin, and the light of the visible region is emitted to Moxifloxacin, thereby having advantages of obtaining the morphological information of the biological tissue without damaging the biological tissue.

Second, the single photon continuous wave light source is used for the fluorescence excitation of Moxifloxacin, and therefore the expensive femtosecond laser device required for two-photon excitation is not needed, thereby having advantages of reducing costs of equipment for taking a imaging.

Third, the fluorescence excitation caused by emitting the single photon continuous wave light to Moxifloxacin has an excitation efficiency higher than that of the two-photon excitation, thereby having advantages of making a general continuous wave (CW) laser device, a light emitting diode (LED) and a discharge lamp (DL) be available, and capturing an image at higher speed than that of the two-photon excitation.

Fourth, the single photon excitation wavelength of the near-ultraviolet region is applied to the excised biological tissue, which is free from cell damage, thereby having advantages of capturing the image of the excised biological tissue at a higher fluorescence excitation efficiency and a higher speed as compared with the two-photon excitation and the visible single photon excitation.

Although a few exemplary embodiments of the present disclosure have been shown and described, it will be appreciated by those skilled in the art that changes may be made in these embodiments without departing from the principles and spirit of the disclosure, the scope of which is defined in the appended claims and their equivalents.

What is claimed is:

1. An imaging method of using fluoroquinolone antibiotics, the method comprising:
staining cells of biological tissue with fluoroquinolone antibiotics;
emitting light from a light source to the biological tissue stained with the fluoroquinolone antibiotics; and
capturing an image of the biological tissue through the fluoroquinolone antibiotics based on fluorescence excitation caused by the excitation light illuminated to the biological tissue,
wherein the light from the light source comprises light for single photon excitation of the fluoroquinolone antibiotics.

2. The imaging method according to claim 1, wherein the fluoroquinolone antibiotics comprise Moxifloxacin.

3. The imaging method according to claim 2, wherein the light from the light source has a continuous wave wavelength range comprising a near-ultraviolet region and a visible region.

4. The imaging method according to claim 3, wherein the near-ultraviolet region and the visible region of the light from the light source range from 300 nm to 476 nm.

5. The imaging method according to claim 1, wherein the biological tissue comprises at least one of external organs, and internal organs, which can be subjected to endoscopy and laparoscopy, of a human body.

6. The imaging method according to claim 5, wherein
the external organs comprise at least one among cornea, skin and tongue, and
the internal organs comprise at least one among small intestine, large intestine, stomach, bladder, brain, lung, esophagus, liver, and pancreas.

7. The imaging method according to claim 1, wherein the capturing the image of the biological tissue comprises:
a photon moving operation in which fluorescence of the fluoroquinolone antibiotics generated by the light emitted to the biological tissue is moved to a light detector;
a photon collecting operation in which the fluorescence moved to the light detector is collected at the light detector;
a photo signal processing operation in which the fluorescence collected at the light detector is subjected to a signal process in a data driving/obtaining board so as to be output through an output section; and
a photon outputting operation in which a fluorescent signal processed in the photon signal processing operation is output through the output section.

8. An imaging device comprising:
a light source configured to emit light to biological tissue stained with fluoroquinolone antibiotics; and
a variable neutral density (ND) filter configured to a penetration amount of light emitted from the light source;
a scanner configured to adjust an angle at which light emitted from the light source or fluorescence of the fluoroquinolone antibiotics excited by the light is reflected;
a dichroic mirror configured to transmit or reflect light in accordance with wavelengths of the light;
a lens configured to control a path via which the light from the light source or the fluorescence light of the fluoroquinolone antibiotics excited by the light travels;
a light detector configured to collect the fluorescence light of fluoroquinolone antibiotics;
a data driving/obtaining board configured to perform a signal process to output the fluorescence collected at the light detector; and
an output section configured to output the fluorescence processed in the data driving/obtaining board,
wherein the light from the light source comprises light for single photon excitation of the fluoroquinolone antibiotics.

9. The imaging device according to claim 8, wherein the fluoroquinolone antibiotics comprise Moxifloxacin.

10. The imaging device according to claim 9, wherein the light from the light source has a continuous wave wavelength range comprising a near-ultraviolet region and a visible region.

11. The imaging device according to claim 10, wherein the near-ultraviolet region and the visible region of the light from the light source range from 300 nm to 476 nm.
